(19) United States
(12) Patent Application Publication
(10) Pub. No.: US 2014/0251332 A1
(43) Pub. Date: Sep. 11, 2014

(54) Ambient Aerosol Manifold For Inline Oxygen Mask

(71) Applicant: Tyson Martin, (US)

(72) Inventor: Tyson Martin, Palm Harbor, FL (US)

(21) Appl. No.: 13/790,528

(22) Filed: Mar. 8, 2013

(51) Int. Cl. A61M 16/06; A61M 16/08; A61M 16/00; A61M 16/14; A61M 16/20

(52) U.S. Cl. CPC A61M 16/0683; A61M 16/14; A61M 16/208; A61M 16/0057; A61M 16/0875
USPC 128/203.29

(57) ABSTRACT

Disclosed is a device whereby a nebulizer may be operatively connected to a non-rebreather oxygen mask so that inhalation therapy treatments may be provided to a patient wearing the mask without compromising the mask's integrity. The invention basically comprises an ambient aerosol manifold that is operatively inserted in fluid-communicating relation between the mask and the mask reservoir. A fitting is provided on the manifold for receiving the exit port of the nebulizer. The fitting, or nebulizer receiver, may be integrally formed on the manifold, or may be removably attachable thereto.

### TECHNICAL FIELD

This disclosure relates to a mechanism for securing a nebulizer to an oxygen mask. More specifically, the present invention relates to an ambient aerosol manifold that may be attached in fluid-communicating relation to a standard non-rebreather oxygen mask. The manifold is inserted between the mask and its reservoir, and a fitting is provided whereby a nebulizer may be attached to the manifold. By virtue of the manifold of the present invention, nebulizer treatments may be administered to a patient without compromising the integrity of the oxygen mask.

### BACKGROUND OF THE INVENTION

Non-rebreather oxygen masks are well-known and commonly used within the medical profession. A “non-rebreather” mask simply means that the patient's exhaled breath freely passes outside the mask so that all breath inhaled comes from the oxygen supply source in order to provide a constant supply of properly oxygen-enriched gas for breathing. This is accomplished by providing a flexible seal around the perimeter of the mask on the patient's face, and one or more one-way valves in the body of the mask that will permit the exhaled breath to pass from the mask, but will not permit ambient air to enter the mask.

Frequently, though certainly not always, when a patient is receiving oxygen-enriched gas for breathing, the administration of inhalation therapy medications may also be required. Such inhalation therapy medications are typically provided with a device known as a nebulizer, which provides treated gasses to the patient during inhalation. Treatment may involve moisture, elevated temperature, medications, or combinations of those treatment modes.

Current state-of-the-art non-rebreather oxygen masks simply do not provide for the administration of inhalation therapy in a convenient, economical, and safe manner that does not compromise the integrity of the non-rebreather oxygen mask. In fact, inhalation therapy treatments with the use of a nebulizer are frequently conducted today by inserting a tube from the nebulizer under the flexible seal around the perimeter of the mask and using tape in an effort to “seal” the mask and the nebulizer tube to the patient so that the integrity of the mask is not compromised during the inhalation therapy treatment provided by the nebulizer.

Not only is this uncomfortable for the patient, but it is clearly subject to a reduced level of patient care in that ambient air can be admitted into the mask if the tape is not properly applied or if the tape fails during treatment.

One example of the background art is U.S. Pat. No. 5,701,886 to Ryatt. Ryatt discloses a treatment non-rebreather assembly and a method for delivering oxygen and medication. Ryatt, however, does not disclose a means of rotatably coupling a nebulizer to an oxygen mask. Nor does Ryatt teach or disclose a fitting for allowing a nebulizer to remain in a generally vertical orientation while in use.

It is therefore clear that a great need remains for a simple, economical, and medically safe means for attaching a nebulizer to a non-rebreather oxygen mask in a fashion that does not compromise the integrity of the seal of the mask to the patient's face.

### SUMMARY OF THE INVENTION

This disclosure provides a mechanism for securing a nebulizer to the oxygen mask of a patient.

The disclosed system has several important advantages. For example, it permits the nebulizer to remain in a generally vertical orientation regardless of the orientation of the patient or the patient's oxygen mask.

A further possible advantage is that respiratory treatments can be delivered to the patient without breaking the seal about the perimeter of the oxygen mask.

Still yet another possible advantage of the present system is to interconnect a nebulizer to an oxygen mask via a rotatable coupling, thereby allowing the angular position of the nebulizer to be adjusted as needed.

Another advantage of the present system is achieved by interconnecting a nebulizer at a position upstream of an oxygen supply port, thereby optimizing the patient's respiratory treatment.

The present invention relates to a device whereby a nebulizer may be operatively connected to a non-rebreather oxygen mask so that inhalation therapy treatments may be provided to a patient wearing the mask without compromising the mask's integrity. The invention basically comprises an ambient aerosol manifold that is operatively inserted in fluid-communicating relation between the mask and the mask reservoir. A fitting is provided on the manifold for receiving the exit port of the nebulizer. The fitting, or nebulizer receiver, may be integrally formed on the manifold, or may be removably attachable thereto.

Various embodiments of the invention may have none, some, or all of these advantages. Other technical advantages of the present invention will be readily apparent to one skilled in the art.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a side view of a standard non-rebreather oxygen mask, with the patient's profile shown in phantom.

FIG. 2 is a view similar to that of FIG. 1 with the addition of the ambient aerosol manifold of this invention shown with the exit port of a standard nebulizer attached thereto.

FIG. 3 is a front elevation of the ambient aerosol manifold of this invention, with the exit port of a standard nebulizer attached thereto.

FIG. 4 is a side view of the invention shown in FIG. 3.

FIG. 5 is a side view of the nebulizer receiver of the present invention.

FIG. 6 is a side view of the top half of the nebulizer exit port.

FIG. 7 is a side view of the bottom half of the nebulizer exit port.

FIG. 8 is a side view of the main fitting of the ambient aerosol manifold of this invention.

FIG. 9 is a depiction of an alternative embodiment of the oxygen mask of the present invention.

Similar reference characters refer to similar parts throughout the several views of the drawings.

PARTS LIST

| | |
|---|---|
| 10 | Aerosol Manifold |
| 12 | Non-rebreather Oxygen Mask |
| 13 | Supply Port on Mask |
| 14 | Patient |
| 18 | Straps |
| 20 | Reservoir |
| 22 | Port |
| 23 | End of Reservoir |
| 24 | Valve |
| 26 | Flexible Seal |
| 28 | Nebulizer Exit Port |
| 30 | Nebulizer Receiver |
| 32 | Main Fitting |
| 34 | Top of Main Fitting |
| 36 | Bottom of Main Fitting |
| 38 | Receiver Port |
| 40 | Main Fitting Tip of Receiver |
| 42 | Bottom Socket |
| 43 | Nebulizer |
| 44 | Nebulizer Valve |
| 46 | Top Half of Nebulizer Exit Port |
| 48 | Bottom Half of Nebulizer Exit Port |
| 52 | Fingers |
| 54 | Tubing |
| 56 | Venturi Valve |

### DETAILED DESCRIPTION OF THE DRAWINGS

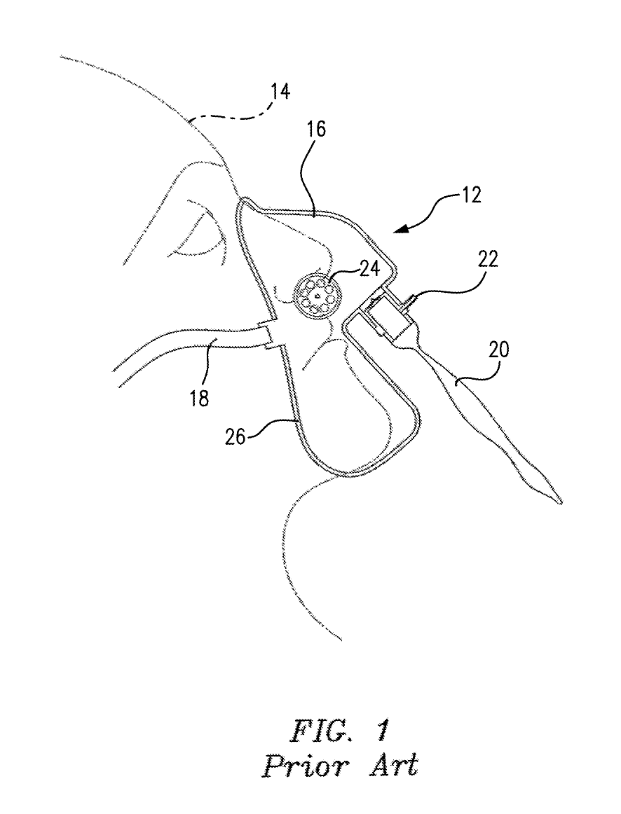

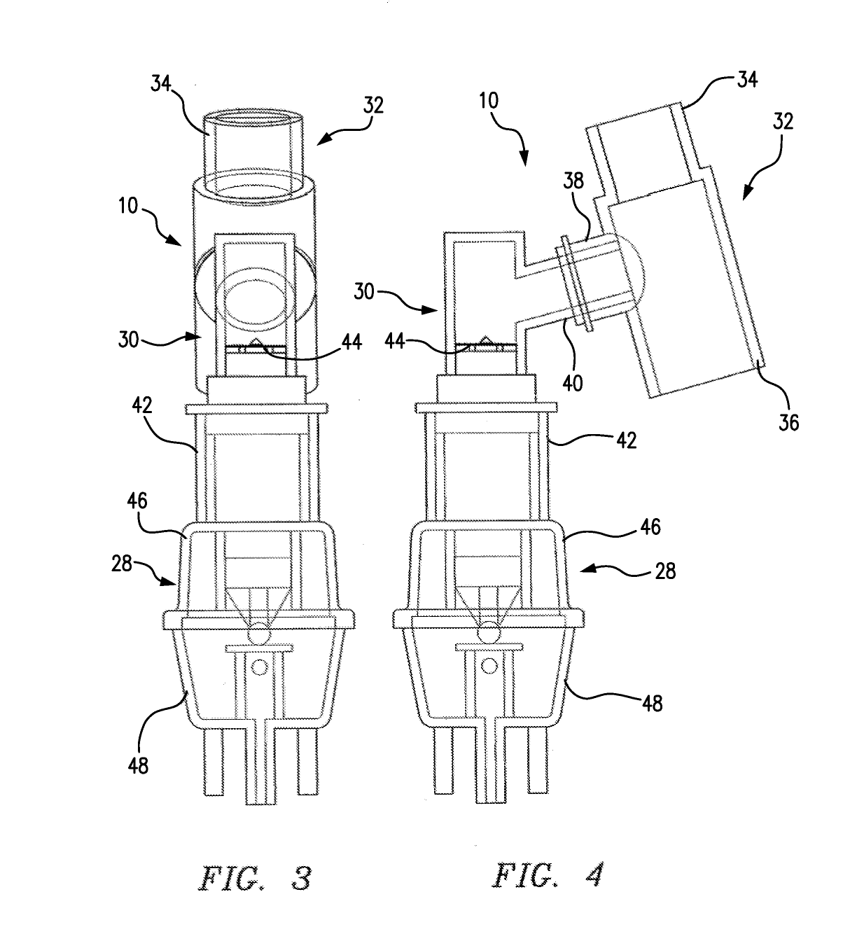

A preferred embodiment of the ambient aerosol manifold of this invention, generally indicated as 10, is shown in the views of FIGS. 3 and 4. However, before turning to a detailed description of that preferred embodiment, attention is invited to the view of FIG. 1.

In FIG. 1, a non-rebreather oxygen mask is generally indicated as 12. Mask 12 is illustrated in operative position on a patient 14 shown in phantom for illustrative purposes. Non-rebreather oxygen mask 12 comprises a mask that is attached to the patient by straps 18 and includes a reservoir 20. Reservoir 20 includes an end portion 23 with a port 22. End portion is adapted to be inserted within a supply port 13 on mask 12. A proper concentration of oxygen is provided to patient 14 through port 22, and breath exhaled by patient 14 passes outwardly from mask 12 through one way valve 24. One way valve 24 is of well-known construction and only permits the passage of material outside mask 12. The substantially airtight fitting of non-rebreather oxygen mask 12 on patient 14 is further accomplished by the construction of mask 12 to further include a flexible seal 26 around its perimeter in contact with patient 14.

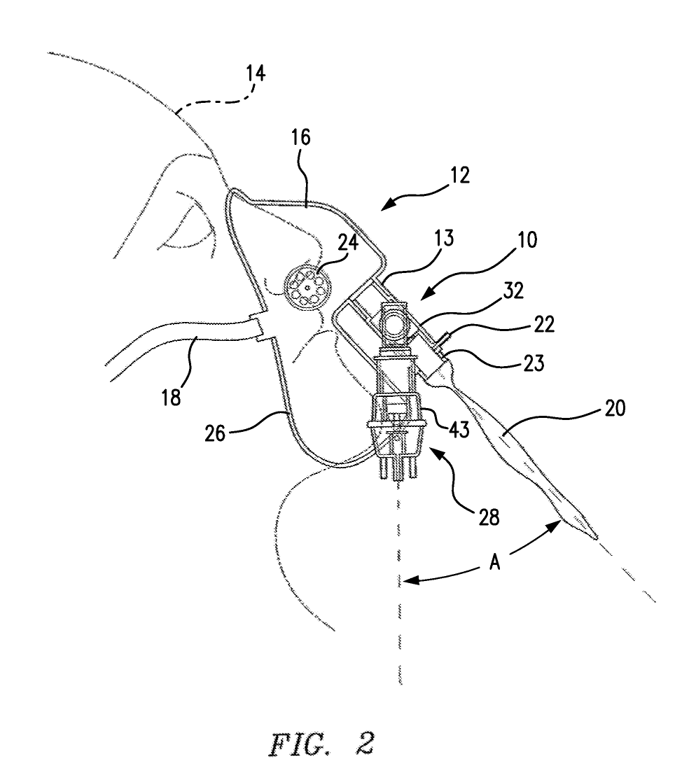

As previously discussed, it is quite common that when a patient 14 is receiving oxygen through a non-rebreather oxygen mask 12 that additional respiratory therapy treatments will be prescribed. Such respiratory therapy treatment is typically provided by the use of a nebulizer, and the primary purpose of the ambient aerosol manifold 10 of 20 this invention is to provide and permit such treatment without compromising the integrity of the non-rebreather oxygen mask 12 on patient 14 during the administration of respiratory therapy. The view of FIG. 2, similar to that of FIG. 1, illustrates operative attachment of the ambient aerosol manifold 10 of this invention to the non-rebreather oxygen mask 12 (of course, it is to be noted that only the nebulizer exit port, generally indicated as 28, is shown, and not an entire nebulizer device).

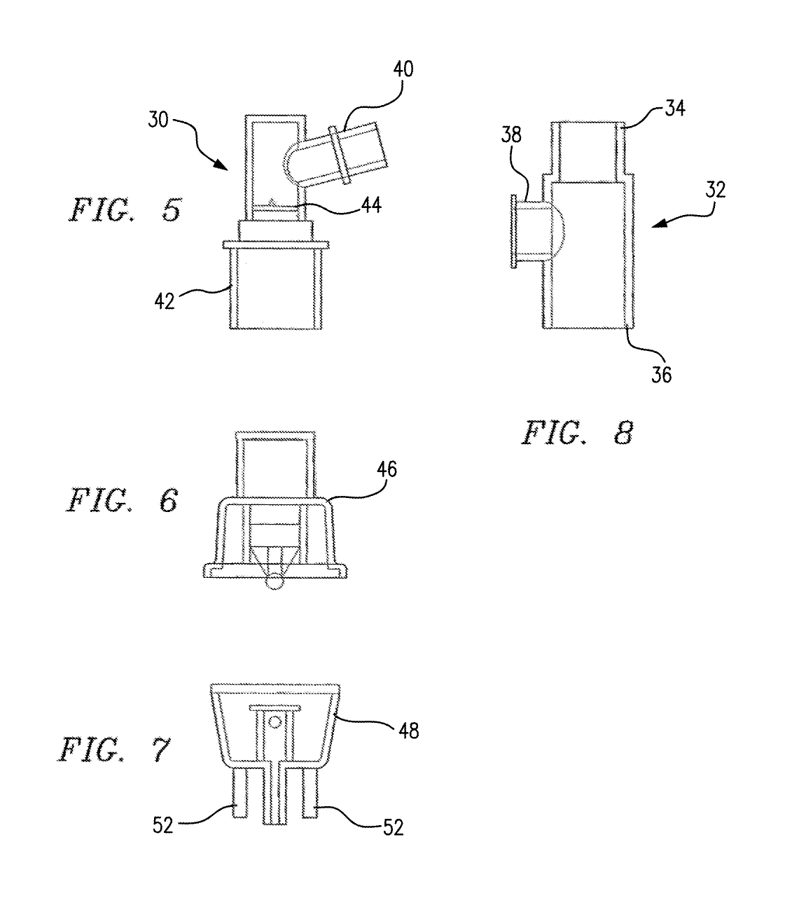

Turning to the views of FIGS. 3 and 4, a preferred embodiment for the ambient aerosol manifold 10 of this invention is shown with the nebulizer exit port 28 attached thereto. As perhaps more clearly seen in the views of FIGS. 5 and 8, ambient aerosol manifold 10 in this preferred embodiment basically comprises a nebulizer receiver, generally indicated as 30, and a main fitting, generally indicated as 32. Main fitting 32 comprises a top 34 that is dimensioned and configured to be received by mask 12, and a bottom 36 that is dimensioned and configured to receive reservoir 20. In this preferred embodiment, main fitting 32 further comprises a receiver port 38 that is dimensioned and configured to receive or accept nebulizer receiver 30, as more fully explained hereinafter. In this preferred embodiment, nebulizer receiver 30 comprises a main fitting tip 40 that is dimensioned and configured to be received by receiver port 38. Nebulizer receiver 30 further comprises a bottom socket 42 dimensioned and configured to receive nebulizer exit port 28 therein, and the nebulizer exit port 28 is typically referred to as an acorn nebulizer.

According to this preferred construction, it should be noted that by virtue of the insertion of main fitting tip 40 into receiver port 38, nebulizer receiver 30 may be rotated with respect to main fitting 32, thereby permitting attachment of the nebulizer device in a position that is not only efficacious, but also comfortable for patient 14.

It is further to be noted that the ambient aerosol manifold 10 of this invention may be constructed such that the nebulizer receiver 30 and the main fitting 32 are unitary, with a direct, permanent connection of nebulizer receiver 30 to main fitting 32. Referring to the views of FIGS. 3 and 4, attention is invited to yet another modification of ambient aerosol manifold 10 to provide another preferred embodiment. As shown by the dotted line, best seen in the view of FIG. 4, nebulizer receiver 30 may comprise a one way nebulizer valve 44 operatively installed between main fitting tip 40 and bottom socket 42 to permit the passage of treatment material from nebulizer exit port 28 through nebulizer receiver 30 and main fitting 32 in only that direction. One way nebulizer valve 44 of this alternate preferred embodiment is of standard, known construction, substantially identical to that of one way valve 24. It is to be understood that the embodiment including a one way nebulizer valve 44 would be appropriate for use in combination with the two-part ambient aerosol manifold 10 shown in the views of FIGS. 5 and 8, as well as in the alternate unitary construction discussed above.

The views of FIGS. 6 and 7 are provided for the purpose of illustrating the standard two-part construction for the nebulizer exit port 28, also referred to as an acorn nebulizer. The view of FIG. 6 illustrates the top half 46 of nebulizer exit port 28, and the view of FIG. 7 illustrates the bottom half 48 of nebulizer exit port 28. These illustrations are provided for the purpose of presenting a clear and complete disclosure of the embodiments of the present invention, and other means for attaching a nebulizer device to the ambient aerosol manifold 10 of this invention might be utilized. Nebulizer exit port 28 optionally includes an additional one way exit port valve at the top of top half 46, and a plurality of fingers 52 are provided at the bottom of bottom half 48 for attachment of the nebulizer device thereto.

The fitting between the nebulizer receiver 30 and the main fitting 32 is preferably a fluid tight, frictional connection. Likewise, main fitting 32 is inserted into the port 13 on mask 12 in a fluid tight, frictional connection. Furthermore, the connection between fitting tip 40 and receiver port 38 is rotatable. This allows receiver 30 as well as nebulizer 43 to remain in a generally vertical orientation when mask 12 and main fitting 32 are inclined (as is usually the case when patient 14 is recumbent). In the most situations, the axis of nebulizer 43 and the axis of main fitting 32 will be at an angle “A,” which will generally be less than 90 degrees.

Although the foregoing embodiment has been described in connection with a non-rebreather mask, it can also be used in connection with other high flow oxygen mask systems. Such high flow oxygen systems can readily be used in connection aerosol mists and/or heated aerosol mists.

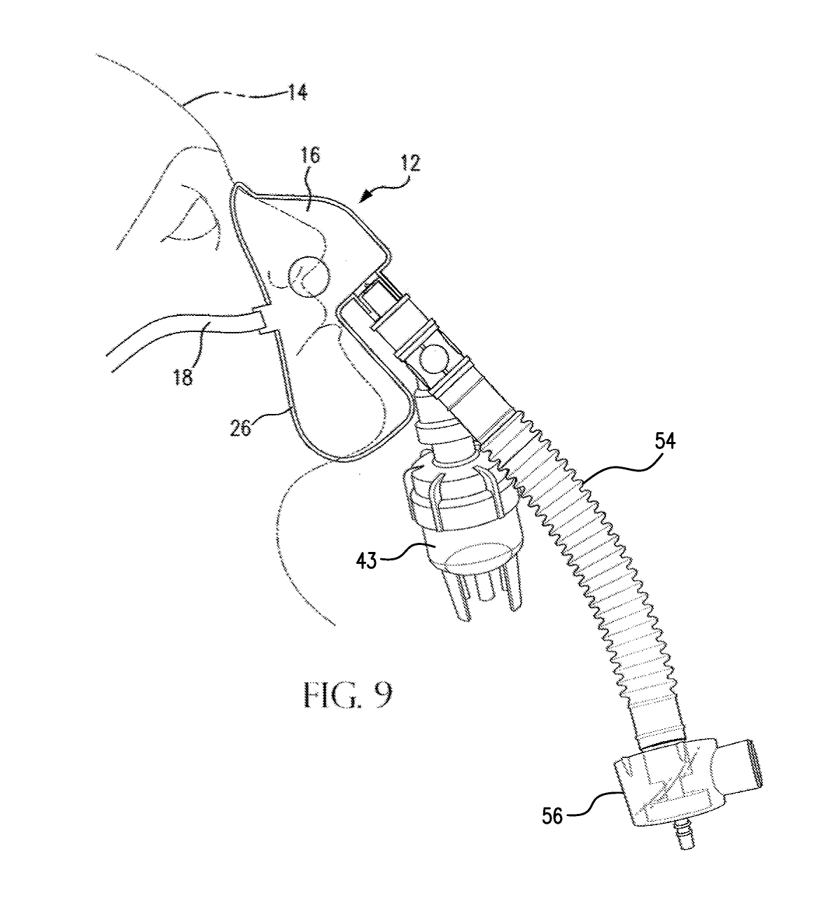

FIG. 9 depicts an alternative embodiment of the present invention. In this embodiment, reservoir 20 is replaced by a length of flexible tubing 54. A venturi valve 56 is secured to the end of the tubing 54 and is utilized in regulating the amount of ambient oxygen the patient inhales. The venturi 56 can be of a known construction. Valves 24 within mask 12 are also eliminated in this embodiment to permit ambient air to enter into mask 12. This embodiment likewise involves a nebulizer 43 as well as a receiver 30 and main fitting 32. As with the primary embodiment described above, this arrangement enables nebulizer 43 to be pivotally fluidly interconnected to mask 12.

Although this disclosure has been described in terms of certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

What is claimed is:

1. A system for delivering respiratory therapy treatments to a patient, the system comprising:
a non-rebreather oxygen mask (12) that is secured over the nose and mouth of the patient (14) via one or more straps (18), the oxygen mask (12) including a one way valve (24) to permit breath exhaled from the patient to exit the oxygen mask (12), the oxygen mask (12) further including a supply port (13) for delivering oxygen to the patient (14);
a main fitting (32) having a top portion (34), a bottom portion (36), and a receiver port (38) located therebetween, the top portion being secured within the supply port (13) via a fluid tight, frictional fit;
a reservoir (20) with an end (23), a port (22) formed within the end (23), the port (22) adapted to be coupled to a supply of oxygen, the end (23) being secured within the bottom portion (36) of the main fitting (32) via a fluid tight, frictional fit;
a nebulizer receiver (30) including an upper end and a bottom socket (42), a fitting tip (40) positioned adjacent the upper end, the fitting tip (40) being received within the receiver port (38) via a fluid tight, frictional fit, a one way valve (44) being positioned within the nebulizer receiver (30);
an acorn-type nebulizer (43) including a top half (46) and a bottom half (48), the top half (46) of the nebulizer (43) being received within the bottom socket (42) of the nebulizer receiver (30) via a fluid tight, friction fit;
whereby the connection between the nebulizer receiver (30) and main fitting (32) is rotatable to permit the nebulizer (43) to remain in a generally vertical orientation when the nebulizer receiver (30) is at an angle.

2. A manifold assembly adapted to be connected to the supply port (13) of an oxygen mask (12), the assembly comprising:
a main fitting (32) having a top portion (34), a bottom portion (36), and a receiver port (38) located therebetween, the top portion being secured within the supply port (13;
a nebulizer receiver (30) including an upper end and a bottom socket (42), a fitting tip (40) positioned adjacent the upper end, the fitting tip (40) being received within the receiver port (38);
a nebulizer (43) including a top half (46) and a bottom half (48), the top half (46) of the nebulizer (43) being received within the bottom socket (42) of the nebulizer receiver (30).

3. The assembly as described in claim 2 wherein the connection between the nebulizer receiver (30) and main fitting (32) is rotatable.

4. The assembly as described in claim 3 wherein the nebulizer (43) and the main fitting (34) each have a longitudinal axis and wherein the longitudinal axis of the nebulizer (43) and the longitudinal axis of the main fitting (34) are at an angle “A.”

5. The assembly as described in claim 4 wherein the angle “A” is generally less than 90 degrees.

6. The assembly as described in claim 2 further comprising a reservoir (20) with an end (23), a port (22) formed within the end (23), the port (22) adapted to be coupled to a supply of oxygen, the end (23) being secured within the bottom portion (36) of the main fitting (32).

7. The assembly as described in claim 2 wherein a one way valve is positioned within the nebulizer receiver.

8. The assembly as described in claim 2 wherein an exit port valve is positioned within the upper half of the nebulizer.

9. The assembly as described in claim 2 wherein a length of tubing (54) is secured to the bottom portion (36) of the main fitting (32).

10. The assembly as described in claim 9 wherein a venturi valve (56) is interconnected to the distal end of tubing (54).
